(19) United States
(12) Patent Application Publication
(10) Pub. No.: US 2007/0225658 A1
(43) Pub. Date: Sep. 27, 2007

(54) Unit Dose Delivery Systems

(76) Inventors: Steven D. Jensen, South Jordan, UT (US); Edwin Pangman, West Jordan, UT (US); Densen Cao, Sandy, UT (US); Robert Larsen, Sandy, UT (US)

Correspondence Address: GEOFFREY E. DOBBIN, PATENT ATTORNEY, 4278 SOUTH 6220 WEST, WEST VALLEY CITY, UT, 84128-6501, US

(21) Appl. No.: 11/277,140

(22) Filed: Mar. 21, 2006

(51) Int. Cl. A61M 5/178

(52) U.S. Cl. 604/212

(57) ABSTRACT

The present invention is a number of single piece unit dose delivery systems. Five related embodiments are disclosed with a number of variations with each embodiment. All embodiments are basically a dispensing system with a containment structure holding a single unit dose of materials for a particular procedure. The containment structure may be either a bulbous tube or a tubular structure attached to a valve body which is also attached to a cannula, of any shape, opposite the structure. A distribution means is located at a tip of the cannula. The valve body may be constructed to block initial communication between the containment structure and the cannula by employing a sealing diaphragm, a plunger apparatus, or by utilizing pull or twist-apart valve structures. Establishing communication is then accomplished by either breaking the diaphragm, as with a needle or other sharp object, or activation the valve, be it a plunger, pull apart or twist-apart variety. Alternatively, no blocking means may be employed and the system may be sealed using an end cap over the distribution means.

### FIELD OF THE INVENTION

The present invention relates to material delivery systems and more particularly relates to specialized, disposable delivery systems containing unit doses of material in a secure, sterile manner with easy access to the contained material when use is desired.

### BACKGROUND OF THE INVENTION

Currently, there are a number of different containers dentists use to hold materials as they apply them to a patient's mouth and teeth. These containers all have the same limitation, that is, they all need 2 separate pieces to complete a delivery. All but one also requires the dentist to go back and forth from a pre-filled container to the dental prep area until sufficient material is delivered. This is very cumbersome.

The first device is a dapen dish, a small piece of plastic in which multiple wells are created. The dentist will drip or pour dental materials from a bottle or other container into these wells. The material is transferred by a brush to the teeth. The amount of material that is transferred is only what can be held on the brush without dripping. The dentist goes back and forth multiple times to deliver sufficient material to the job.

In another two-part system, single use cartridges are loaded into a gun apparatus. The gun must be loaded every time a cartridge is emptied of material, which is an extra step, and the cartridges are cumbersome to load. This system also suffers from cross-contamination issues since the same gun goes into every patient's mouth. Therefore, the gun is exposed to a first patient's microbes and must be sterilized before being used on a second patient.

There is also a two-piece unit dose system, basically a brush in pre-filled container. It is a system that does not cross contaminate, since it is thrown away after a single patient's use. The brush is sealed in a pre-filled container and the dentist must break the seal and while holding the brush in one hand and the container in the other. The dentist then does the cumbersome dance back and forth between tooth and container.

Syringes with specialized tips are an improvement over all the other delivery systems. A pre-filled syringe is capable of delivering sufficient material to the prep site without having to go back and forth from a secondary container. The disadvantage of a syringe is the added step of removing the cap, followed by the added step of adding a tip of choice. These are cumbersome steps, especially while wearing latex gloves. The biggest disadvantage of a syringe is cross-contamination. Current syringes contain multiple doses; therefore, they can be used on one or more patients. This allows for the contamination of the syringe and the possibility of cross-contaminating patients with each others' microbes. To overcome this problem, companies have developed little bags or sleeves that can be placed over the end of the syringe which is a huge annoyance and another step in the delivery process.

What is needed, then, is a disposable unit dose system that can deliver all of its pre-filled contents directly to the work site. The system must also be designed as one single unit, so a single piece is all that is needed to deliver material to the work site. Such a system would have no chance of cross-contamination and would eliminate intermediate material transference steps. An added design element would be the use of a single action valve that would keep the material contents isolated and could be activated with minimal operation.

### SUMMARY OF THE INVENTION

In view of the foregoing disadvantages inherent in the known types of delivery systems for dental materials, this invention provides an improved material delivery system. As such, the present invention's general purpose is to provide a new and improved delivery system that is capable of single-handed delivery while simultaneously being a unit dose and disposable after use on a patient. Presently, this invention has four separate embodiments.

The first embodiment, a hermetically sealed puncture valve unit dose system, is best described as a polymer sealed container that contains an internal puncture pin and an extended cannula with brush-tipped end. The device is activated by pressing and forcing the sharp pin through the polymer wall and creating a hole or a flap. Material is then expressed through this hole out the cannula and onto the brush for precise delivery in the oral cavity.

A push button valve unit dose system is a second embodiment. It is best described as a container with a push button valve between the cannula and the container. When the button is depressed, a void is exposed between the container and cannula thus allowing flow of the material to the brush end.

The threaded twist valve unit dose system is best described as a friction/pressure valve that rotates around an axis. When the container and cannula are twisted counter to each other the valve will open one way and close the other direction. When the valve is open it allows material to flow down the cannula. Also, a pull-apart, sliding unit dose system is best described as a friction valve that opens and closes on a single axis by pulling or pushing the internal valve together or apart. In either case, the valve exposes the contents to the cannula, allowing the contents to be expressed out of the unit.

All of these unit dose systems are all one piece systems and have simple valves with which to open the container. A dentist with latex gloves will appreciate the ease of use and the single handed delivery of all the material to the work site. This unit dose will also work well filled with paint for touch-ups or in medicine as a means to deliver and paint an area with medications. Practically any liquid that requires a unit dose system can be loaded into these systems. Likewise, the cannula can optionally be made of a material that will allow it to bend into a desired curve and retain its bent shape. This allows the dentist to customize the shape to the circumstances at the time.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims. Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective view of a preferred embodiment of the invention.

FIG. 2 is an exploded side plan view of a second embodiment of the invention.

FIG. 3 is an exploded perspective view of the embodiment of FIG. 2.

FIG. 4 is an exploded perspective view of a third embodiment of the invention.

FIG. 5 is an exploded top plan view of the embodiment of FIG. 4.

FIG. 6 is an exploded perspective view of a fourth embodiment of the invention.

FIG. 7 is an exploded side plan view of the embodiment of FIG. 6.

FIG. 8 is a top plan view of the embodiment of FIG. 6.

FIG. 9 is an exploded sectional view of the embodiment of FIG. 8, taken along line A-A.

FIG. 10 is an exploded side plan view of a fifth embodiment of the invention.

FIG. 11 is an exploded perspective view of the embodiment of FIG. 10.

FIG. 12 is a sectional view of the embodiment of FIG. 10, taken along line A-A.

FIG. 13 is a side plan view of the embodiment of FIG. 10, with the valve closed.

FIG. 14 is a sectional view of the embodiment of FIG. 13, taken along line B-B.

FIG. 15 is an enlarged view of the embodiment of FIG. 14, taken within circle C.

FIG. 16 is a side plan view of the embodiment of FIG. 10, with the valve open.

FIG. 17 is a sectional view of the embodiment of FIG. 16, taken along line D-D.

FIG. 18 is an enlarged view of the embodiment of FIG. 17, taken within circle E.

FIG. 19 is a side plan view of the embodiment of the invention depicted in FIG. 1.

FIG. 20 is a sectional view of the embodiment of FIG. 14, taken along line F-F.

FIG. 21 is an enlarged view of the embodiment of FIG. 15, taken within circle G.

### DETAILED DESCRIPTION OF THE PREFERRED EMBODIMENTS

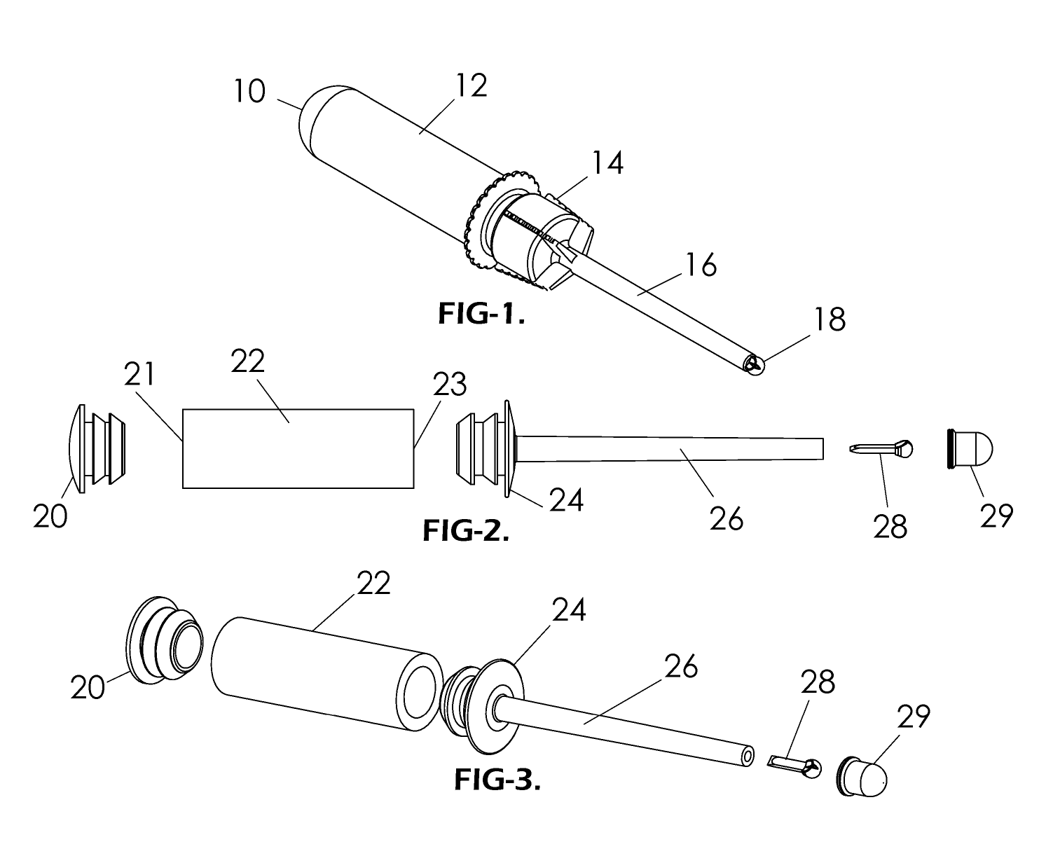

With reference now to the drawings, the various embodiments of the unit dose systems, with specific emphasis on certain preferred embodiments, are herein described. As seen in FIG. 1, the Single Piece Unit Dose Delivery Systems of the present invention comprise a containment structure 12 attached to a cannula 16 via a valve body or other joining means 14. At the open end of cannula 16 is a distribution or spreading means 18. This spreading means is usually termed a “brush”, though various Fibrous flocking means may be employed, an example would be U.S. Pat. No. 6,390,817 to Jensen (2002), utilizing a fibrous flocked applicator. The “brush” may be any type of means known or later developed in the art for the purpose of applying and spreading material onto a patient's teeth and this application will use the term “brush” to include such different means.

Generally, material is loaded into the containment structure 12 and joining means 14 is attached, forming the unit dose system. As shown in FIG. 1, the containment structure is a bulbous tube 12 with a closed end 10 and an open end for receiving the valve body 14. A second embodiment of the containment structure is depicted in FIG. 2. In this second embodiment, the containment structure utilizes a tubular body 22 with openings 21, 23. Hind opening 21 is closed with end cap 20 while fore opening 23 is closed with valve body 24. The end cap 20 and valve body 24 are barbed to secure the interface of the end cap 20 and valve body 24 to the tubular body 22. Either the tubular system of FIG. 2 or the bulbous tube system of FIG. 1 may be utilized without departing from the scope of the invention. Both embodiments are to be understood to be included when using the term “containment structure” unless context clearly dictates otherwise. In this application, all other embodiments of the unit dose systems will be depicted with the tubular construction, though the bulbous tube is equally preferred. In any embodiment, the containment structure 12 is squeezed in order to force contents through the valve body 14 into the cannula 16 and out by brush 18. Therefore, the plasticity of the containment structure must be sufficient to allow total collapse of the structure while being sufficient to withstand storage. The preferred embodiment utilizes a thin sleeve of Polyvinyl Acetate (PVA) with a measured hardness between 40 and 80 durometers, though any other material of sufficient durability and plasticity will suffice. The remaining pieces may be made of any other suitable material, like rigid or rubberized plastics.

In a second embodiment of the unit dose systems, shown In FIGS. 2 and 3, the valve body 24 is open and the entire system is sealed with brush cap 29 placed over brush 28. This embodiment features an easy opening on the system for expressing the material contents.

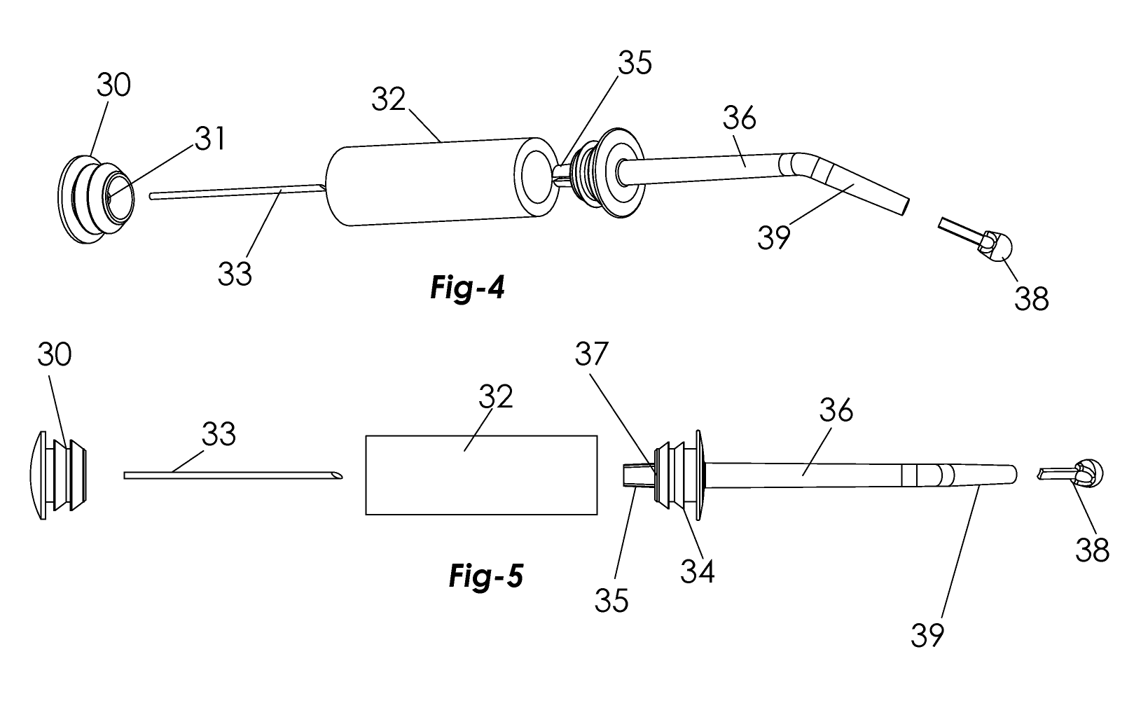

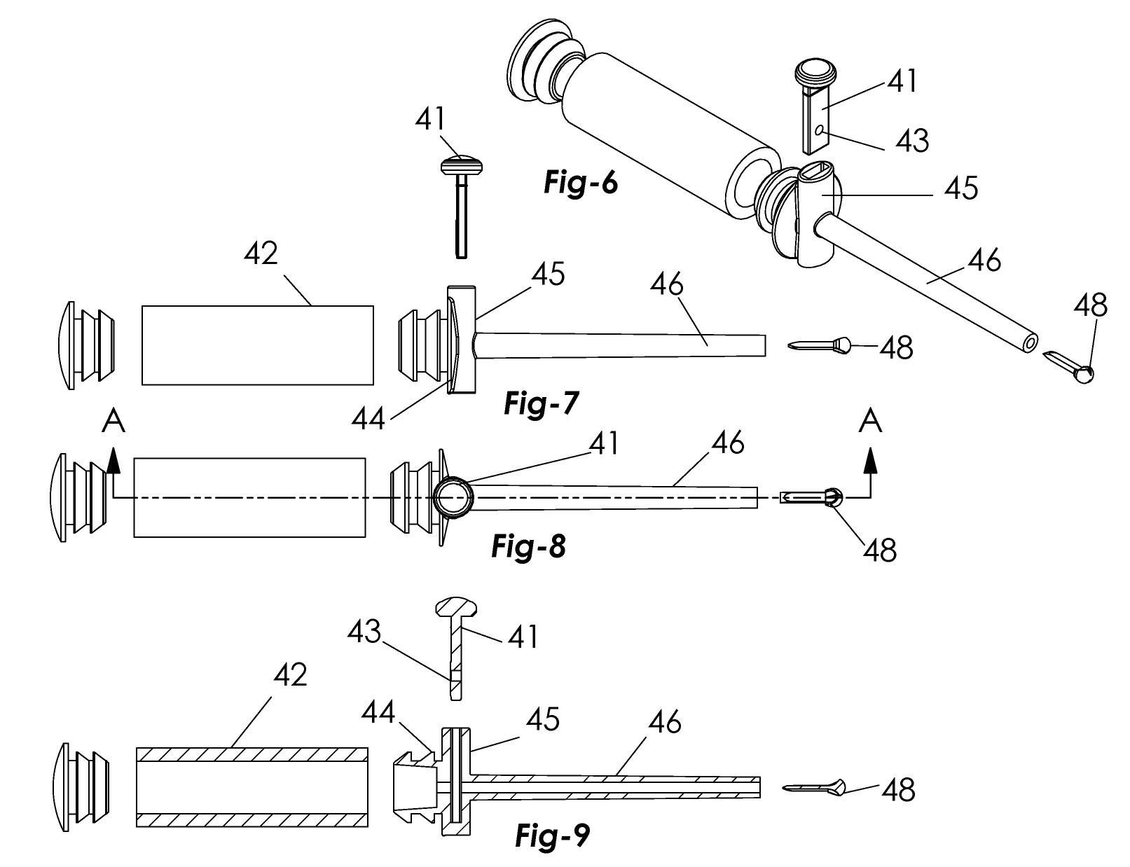

In a third embodiment, shown in FIGS. 4 and 5, valve body 34 contains a sealing diaphragm 37 positioned between the cannula 36 and the containment structure 32. End cap 30 is modified to hold a needle 33 in a seat 31. When the system is assembled the sharp end of needle 33 is nestled between guards 35 on valve body 34 and proximate diaphragm 37. To access the loaded material, the user simply pressed end cap 35 so that needle 33 punctures diaphragm 37, allowing material to flow through valve body 34 into cannula 36. In these figures, cannula 36 has a bent tip 39. The bent tip 39 may be preferred by some users as a matter of access and view while working on, as an example, a patient's mouth. Bent tip 39 may be incorporated in any embodiment of the invention described herein, though not depicted on any other figures and the appearance of bent tip 39 only in FIGS. 4 and 5 should not be understood as limiting.

In a fourth embodiment, valve plunger 41 is inserted into chamber 45 between the cannula 46 and valve body 44. Valve plunger normally rests in chamber 45 blocking communication between containment structure 42, valve body 44 and cannula 46. However, when the valve plunger 41 is depressed into the chamber 45, access hole 43 is positioned into the plane of the cannula 46 and valve body 44, allowing communication and flow of contents through valve body 44 into cannula 46. Chamber 45 is ideally constructed to in some way block button structure 41 from passing entirely through chamber 45 without purposeful force or exiting chamber 45 when initially inserted, a ridge or detent structure, not shown, would provide such capabilities. The opposite motion of valve plunger 41 may also be utilized (i.e. opening the plunger by pulling it out) by adjustment of the position of access hole 43 relative to the rest of the valve plunger.

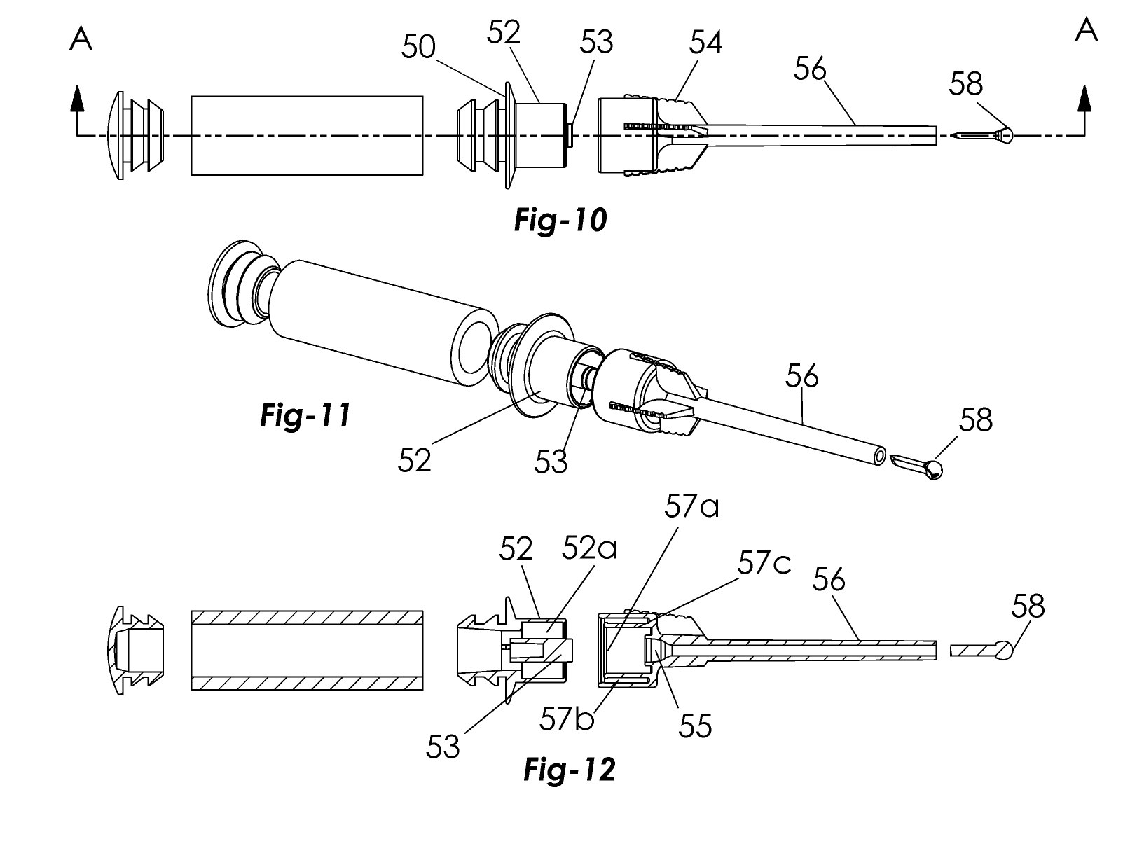

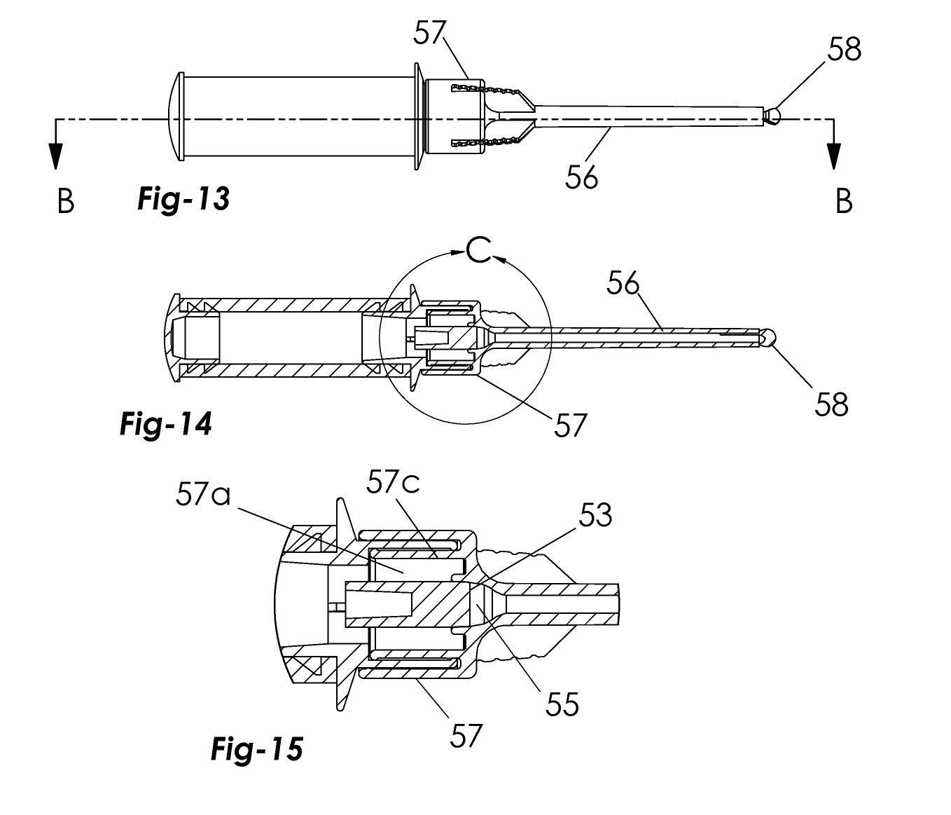

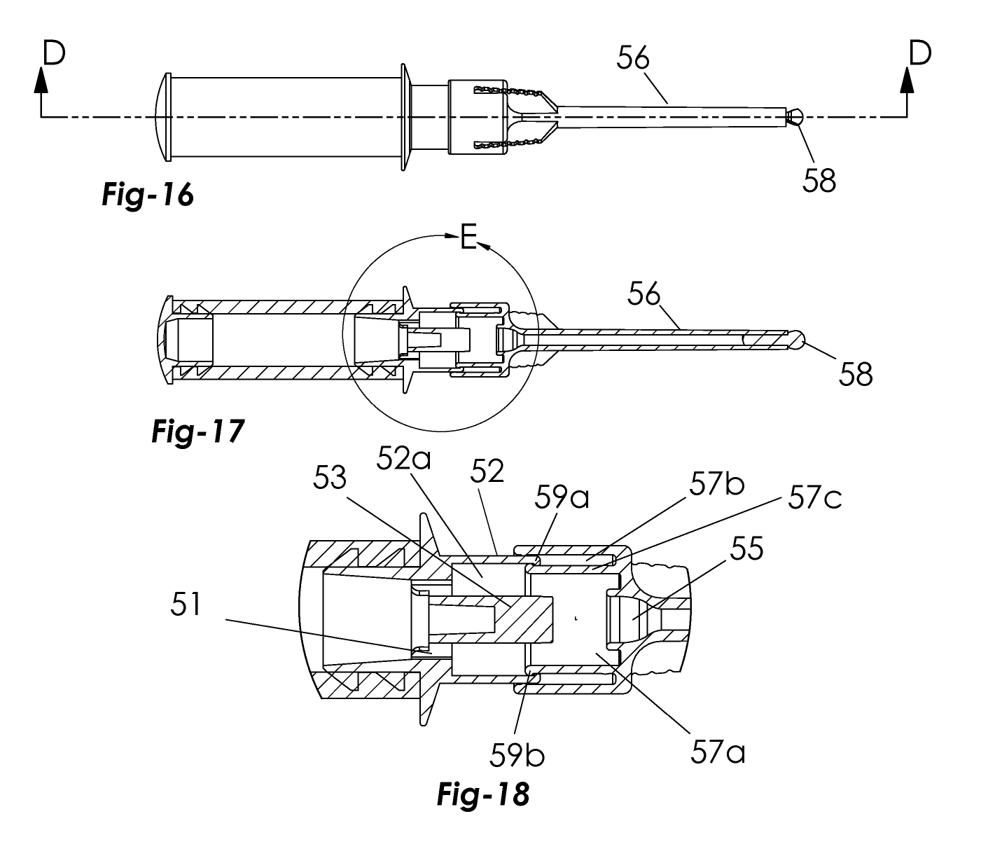

In a fifth embodiment, shown in FIGS. 10-18, the system utilizes a pull-out design for the valve structure. Valve base 50 is nested into the containment structure and its exterior end presents an outer cylindrical wall 52 and central cylindrical pillar 53. Base chamber 57a, the space between stop 53 and wall 52, has communication with the contents of the containment structure through orifice 51 and is open at its furthest end. Valve cover 54 is attached to cannula 56 as previous valve structures were depicted. Inside valve cover 54 is a centrally located valve seat 55, the base of cannula 56, and a coaxial cylindrical cover wall 57 defining inner and outer chambers 57a, 57b. The position of cover wall 57 is such that outer chamber 57b will interface with base wall 52 and cylindrical pillar 53 will rest in valve seat 55 when the valve structure is closed. When pulled into the open position, chamber 52a has fluid communication with inner chamber 57a so that contents of the containment structure may pass though orifice 51, into base chamber 52a and further into inner chamber 57a and out valve seat 55 into cannula 56. Movement termination, so the valve structure is not pulled apart, is accomplished through two interfacing annular rings 59a, 59b, on the base wall 52 and inner wall 57c. These rings should have complimentary angled surfaces to allow the parts to be put together while simultaneously preventing disassembly.

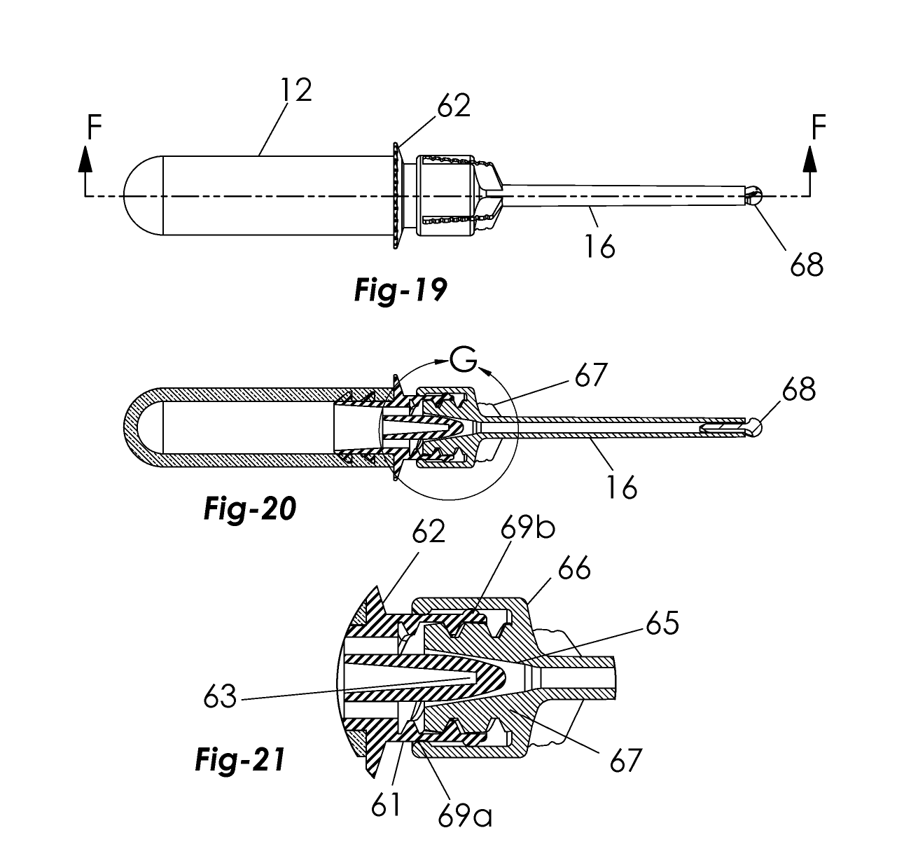

The first depicted embodiment from FIG. 1 is a twist open embodiment and is shown in greater detail in FIGS. 19-21. This embodiment is similar to the immediately previous embodiment in that the valve structure 62 is made of two interfacing parts, the valve base 62 and valve cover 64. Valve base 62 comprises an outer wall 61 and an inner, tapering pylon 63 while valve cover 64 comprises an outer wall 66 and an inner wall 67 with a tapering inner circumference that defines a valve seat 65 open to cannula 16. Walls 61 and 67 are threaded for a mating interface and present annular rings 69a, 69b to prevent separation. In this embodiment, the user simply twists open the valve body 62 and fluid communication is established around pylon 63, through valve seat 65 and into cannula 16. Initial sealing of both the first and last embodiments may be accomplished through means known in the art to prevent accidental twisting and pulling of caps and the like, including sealing rings, shrink wrapping, protective labels, break-off tabs, and any other means known or later developed in the art.

Although the present invention has been described with reference to preferred embodiments, numerous modifications and variations can be made and still the result will come within the scope of the invention. Such modifications include increasing or decreasing viscosity and peroxide concentration for various purposes. No limitation with respect to the specific embodiments disclosed herein is intended or should be inferred.

What is claimed is:

1. A unit dose delivery system comprising:
A compressible material containment structure defining an interior;
A valve body at one position on the containment structure;
A cannula, opposite the valve body from the containment structure and with a means of communication through the valve body with the interior of said containment structure, an end of the cannula opposite the valve body being defined as a tip; and
A distribution means located on the tip of the cannula;
Wherein the containment structure has a volume sufficient to contain material for a single application of said material.

2. The system of claim 1, the containment structure comprising a tubular body sealed on one end with a barbed end cap and sealed on a second end with the valve body, the valve body likewise being barbed.

3. The system of claim 2, further comprising a cannula cap, positioned over the distribution means.

4. The system of claim 1, the containment structure comprising a compressible bulbous tube, having a single open end, said valve body being barbed to secure the bulbous tube to the valve body at the open end.

5. The system of claim 4, further comprising a cannula cap, positioned over the distribution means.

6. The system of claim 1, further comprising a cannula cap, positioned over the distribution means.

7. The system of claim 1, the means of communication being initially closed and the system further comprising a means to open the means of communication.

8. The system of claim 7, the containment structure comprising a tubular body sealed on one end with a barbed end cap and sealed on a second end with the valve body, the valve body likewise being barbed.

9. The system of claim 7, the containment structure comprising a compressible bulbous tube, having a single open end, said valve body being barbed to secure the bulbous tube to the valve body at the open end.

10. The system of claim 1, the system further comprising:
the containment structure still further comprising:
a tubular body sealed on one end with a barbed end cap and sealed on a second end with the valve body, the valve body likewise being barbed;
a needle seat axially located relative to the tubular body on an interior side of the end cap; and
a needle, having a sharp end and a second end, the second end being seated within the needle seat and the needle having a length slightly shorter than a length defined by an interior of the containment structure when assembled; and
the valve body still further comprising:
a diaphragm sealing the means of communication; and
at least one needle guard, coaxial with the means of communication;
wherein the assembled system has the sharp end of the needle positioned proximate the diaphragm and within the needle guard such that compression on the end cap drives the needle into the diaphragm, opening the means of communication.

11. The system of claim 1, the system further comprising:
the containment structure still further comprising:
a bulbous tube having one open end, said open end sealed with the valve body, the valve body being barbed;
a needle seat axially located on an opposite side of the valve body; and
a needle, having a sharp end and a second end, the second end being seated within the needle seat and the needle having a length slightly shorter than a length defined by an interior of the containment structure when assembled; and
the valve body still further comprising:
a diaphragm sealing the means of communication; and
at least one needle guard, coaxial with the means of communication;
wherein the assembled system has the sharp end of the needle positioned proximate the diaphragm and within the needle guard such that compression on the bulbous tube towards the valve body drives the needle into the diaphragm, opening the means of communication.

12. The system of claim 1, the valve body further comprising:
A valve chamber located between the cannula and the remainder of the valve body, said chamber allowing communication between the containment structure and the cannula and also having an opening to an exterior of the valve body and a valve plunger, and said valve plunger further comprising:
A button surface;
A plunger wall, slidable within the opening of valve chamber, thereby sealing the same and blocking communication between the containment structure and the cannula while in a first position; and
An access orifice, positioned on the plunger wall such that communication between the containment structure and cannula is allowed through the access orifice when the valve plunger is in a second position.

13. The system of claim 12, the containment structure comprising a tubular body sealed on one end with a barbed end cap and sealed on a second end with the valve body, the valve body likewise being barbed.

14. The system of claim 12, the containment structure comprising a compressible bulbous tube, having a single open end, said valve body being barbed to secure the bulbous tube to the valve body at the open end.

15. The system of claim 1, the valve body comprising:
A valve base further comprising:
A barbed end inserted within the containment structure and an exterior end, said exterior end being still further comprised of a cylindrical wall and a pillar, both being coaxial with the valve base and defining an inner chamber, and al least one orifice allowing communication between the containment structure and the inner chamber; and
A valve cover, attached to the cannula, said valve cover further comprising:
An outer cylindrical wall, extending opposite the cannula;
An inner cylindrical wall, extending opposite the cannula and defining both an interface chamber of similar width as the cylindrical wall of the valve base between it and the outer wall and an interior in general;
A valve seat, inside the interior of the valve cover and both open to and opposite the cannula; and
a retention means;
wherein the valve base is inserted within the valve cover, the cylindrical wall nesting within the interface chamber and the pillar resting within the valve seat when in a closed position and the valve base and valve cover are pulled apart, to the point the restraining means allows, such that the valve cover is not removed and the pillar is removed from the valve seat, thereby establishing communication between the containment structure and the cannula.

16. The system of claim 15, the containment structure comprising a tubular body sealed on one end with a barbed end cap and sealed on a second end with the valve body, the valve body likewise being barbed.

17. The system of claim 15, the containment structure comprising a compressible bulbous tube, having a single open end, said valve body being barbed to secure the bulbous tube to the valve body at the open end.

18. The system of claim 1, the valve body comprising:
A valve base further comprising:
A barbed end inserted within the containment structure and an exterior end, said exterior end being still further comprised of a threaded cylindrical wall and a pylon, both being coaxial with the valve base and defining an inner chamber, and al least one orifice allowing communication between the containment structure and the inner chamber; and
A valve cover, attached to the cannula, said valve cover further comprising:
An outer cylindrical wall, extending opposite the cannula;
An inner cylindrical wall, extending opposite the cannula and threaded to interface with the cylindrical wall of the valve base, both valve cover walls defining an interior;
A valve seat, inside the interior of the valve cover and both open to and opposite the cannula; and
a retention means;
wherein the valve base is inserted within the valve cover, the cylindrical wall nesting within the interface chamber and the pylon resting within the valve seat when in a closed position and the valve base and valve cover are twisted apart, to the point the restraining means allows, such that the valve cover is not removed and the pylon is removed from the valve seat, thereby establishing communication between the containment structure and the cannula.

19. The system of claim 18, the containment structure comprising a tubular body sealed on one end with a barbed end cap and sealed on a second end with the valve body, the valve body likewise being barbed.

20. The system of claim 18, the containment structure comprising a compressible bulbous tube, having a single open end, said valve body being barbed to secure the bulbous tube to the valve body at the open end.
